(19) United States
(12) Patent Application Publication
(10) Pub. No.: US 2007/0104669 A1
(43) Pub. Date: May 10, 2007

(54) Cosmetic composition comprising at least one ester, at least one acrylic polymer, at least one cyclodextrin and at least one surfactant, and uses thereof

(76) Inventor: Rainer Muller, Leopoldshafen (DE)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/585,978

(22) Filed: Oct. 25, 2006

Related U.S. Application Data
(60) Provisional application No. 60/731,268, filed on Oct. 31, 2005.

(30) Foreign Application Priority Data
Oct. 26, 2005 (FR) 05 53256

(51) Int. Cl. A61K 8/81; A61K 8/73; A61K 8/37; A61K 8/36

(52) U.S. Cl. 424/70.13; 424/70.16; 424/70.31

(57) ABSTRACT

Disclosed herein are compositions comprising, in a cosmetically acceptable aqueous medium, at least one anionic surfactant, at least one liquid ester of a carboxylic acid comprising from 4 to 6 carbon atoms and of an alcohol comprising from 12 to 26 carbon atoms, at least one compound chosen from cyclodextrins and derivatives thereof, and at least one acrylic thickening polymer. Also disclosed herein are compositions further comprising at least one conditioning agent. The compositions according to the present disclosure may be used, in at least one embodiment, as shampoos.

This application claims benefit of U.S. Provisional Application No. 60/731,268, filed Oct. 31, 2005, the contents of which are incorporated herein by reference. This application also claims benefit of priority under 35 U.S.C. §119 to French Patent Application No. FR 05 53256, filed Oct. 26, 2005, the contents of which are also incorporated herein by reference.

Disclosed herein are cosmetic compositions (for example, for topical use) comprising, in a cosmetically acceptable medium, at least one cyclodextrin, at least one anionic surfactant, at least one fatty ester and at least one acrylic thickening polymer. Also disclosed herein are compositions further comprising at least one conditioning agent.

It is known that hair that has been sensitized (i.e. damaged and/or embrittled) to varying degrees due to the action of atmospheric agents and/or mechanical and/or chemical treatments, such as dyeing, bleaching, and/or permanent-waving, is often difficult to disentangle and to style, and lacks softness.

It is further known to use conditioning agents, for instance, insoluble conditioning agents, in compositions for washing and/or caring for keratin materials such as the hair, in order to facilitate the disentangling of the hair and to give it softness, sheen, and suppleness.

Given the insoluble nature of certain conditioning agents, for instance, silicones and oils, it is desirable to maintain the conditioning agents in uniform dispersion in the medium without, however, causing a reduction in the viscosity and the detergent and foaming properties of the compositions. Conditioning agents, such as silicones and oils, are generally conveyed onto the treated keratin materials in order to give them, depending on the application, softness, sheen, and/or disentangling properties.

It is also known that products, for example, cosmetic products, having an iridescent, moiré, or metallized appearance and/or effect are widely appreciated by consumers for their attractive appearance, giving the product a look of richness. The agents that afford this effect are nacreous agents or nacres generally comprising crystals that remain dispersed in the compositions and reflect light.

Long-chain ester derivatives are commonly used to make compositions, such as cosmetic compositions, nacreous. However, these derivatives may have crystallization problems that may result in a change in the viscosity of the compositions over time.

Long-chain ether and thioether derivatives such as those described in European Patent Application Nos. 457 688 and WO 98/03155 are also known. However, these agents may opacify the compositions without, or without sufficiently, giving them a nacreous effect.

It has also been found that, on account of their low density, these nacreous agents often have the drawback of rising to the surface of the shampoo and forming a layer thereon that consumers find unattractive.

Furthermore, in certain cases, these fatty-chain compounds have the drawback of giving a laden feel to the hair and a lack of lightness and volume to the hairstyle.

Moreover, all these nacreous agents are water-insoluble compounds and have a melting point above 50° C. To manufacture nacreous compositions, the compositions thus need to be heated above the melting point of the nacreous compound and then cooled, and the other compounds of the composition are subsequently added. In order to reduce the energy consumption and to reduce the manufacturing time, it is desirable to prepare the compositions without heating.

Intemational Patent Application Publication No. WO 03/088934 describes the use of cyclodextrin as a nacreous agent that can be used without heating. However, compositions containing a cyclodextrin are still not sufficiently stable.

Stabilizers such as crosslinked acrylic polymers of the Carbopol type are frequently used to thicken and stabilize cosmetic compositions containing insoluble conditioning agents. However, these stabilizers are not always sufficient to ensure good stability on storage over time, especially at high temperature (45° C.).

The present inventor has discovered that it is possible to formulate cosmetic compositions for treating keratin materials, for example, shampoos, which have a nacreous appearance, while at the same time having the desired aesthetic and cosmetic properties, by using in these cosmetic compositions at least one cyclodextrin or a derivative thereof, at least one surfactant, at least one fatty ester, and at least one acrylic polymer.

Disclosed herein, therefore, are compositions, for example, cosmetic compositions, comprising, in a physiologically acceptable, and in at least one embodiment, a cosmetically acceptable, aqueous medium, at least one anionic surfactant, at least one liquid ester of a carboxylic acid comprising from 4 to 6 carbon atoms and of an alcohol comprising from 12 to 26 carbon atoms, at least one cyclodextrin or a derivative thereof, and at least one acrylic thickening polymer.

The compositions may show very good homogeneity and good stability of the nacre, and also a viscosity that is satisfactory for application to keratin materials. There is, in at least one embodiment, no phase separation over time of the cyclodextrin and/or of the insoluble conditioning agents.

Further, in at least one embodiment, no uncontrolled graining-out or thickening of the composition over time takes place. Finally, the compositions may have a non-runny, fondant texture. The lather may be easy to rinse out.

Also disclosed herein is a washing and/or conditioning process comprising applying a composition of the present disclosure to keratin materials.

Further disclosed herein is a process for improving the stability on storage of a cosmetic composition comprising at least one cyclodextrin and at least one anionic surfactant, the process comprising adding to the composition at least one acrylic polymer and at least one liquid ester of a carboxylic acid comprising from 4 to 6 carbon atoms and of an alcohol comprising from 12 to 26 carbon atoms.

Other subjects and aspects of the present disclosure will become apparent on reading the description and the examples that follow.

As used herein, the term “keratin materials” means the hair, the eyelashes, the eyebrows, the skin, the nails, mucous membranes, and/or the scalp, and in at least one embodiment, the hair.

As used herein, the terms “nacreous agent” and “nacre” mean an agent that produces a nacreous, iridescent, moire, or metallized appearance and/or effect.

Acrylic Thickening Polymers

The compositions of the present disclosure comprise at least one acrylic thickening polymer.

As used herein, the term “acrylic polymer” means a polymer resulting from the polymerization of at least one monomer having the structure:

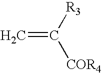

wherein:
R₃ is chosen from hydrogen and linear or branched C₁-C₄ alkyl radicals,
R₄ is chosen from hydrogen, linear or branched C₁-C₄ alkyl radicals, NR₅R₆ radicals, and linear or branched C₁-C₃₀ alkoxy radicals, optionally substituted with at least one radical chosen from hydroxyl radicals and quaternary ammonium radicals, and
R₅ and R₆ are chosen from hydrogen and optionally oxyalkylenated C₁-C₃₀ alkyl radicals, the alkyl radicals optionally comprising a sulfonic group.

In at least one embodiment, R₃ may be chosen from hydrogen and methyl radicals.

As used herein, the term “thickening polymer” means a polymer having, as a 1% by weight solution or dispersion of active material in water or in ethanol at 25° C., a viscosity of greater than 0.2 poise at a shear rate of 1 s⁻¹. The viscosity is measured with a Haake RS600 viscometer from Thermo Electron. This viscometer is a controlled-stress viscometer with cone-plate geometry (for example of diameter 60 mm).

The thickening polymers may be chosen, for example, from:
(a) acrylic associative thickeners;
(b) crosslinked acrylic acid homopolymers;
(c) crosslinked copolymers of (meth)acrylic acid and of a (C₁-C₆)alkyl acrylate;
(d) nonionic homopolymers and copolymers containing ethylenically unsaturated monomers of ester and/or amide type;
(e) ammonium acrylate homopolymers and copolymers of ammonium acrylate and of acrylamide;
(f) (meth)acrylamido(C₁-C₄)alkylsulfonic acid homopolymers and copolymers; and
(g) crosslinked methacryloyl(C₁-C₄)alkyltri(C₁-C₄)alkylammonium homopolymers and copolymers.

As used herein, the expression “associative thickeners” means amphiphilic thickeners comprising both hydrophilic units and hydrophobic units, for example, comprising at least one C₈-C₃₀ fatty chain and at least one hydrophilic unit.

(a) Acrylic associative thickeners that may be used according to the present disclosure include, but are not limited to, acrylic associative polymers chosen from:
(i) nonionic amphiphilic polymers comprising at least one fatty chain and at least one hydrophilic unit;
(ii) anionic amphiphilic polymers comprising at least one hydrophilic unit and at least one fatty-chain unit;
(iii) cationic amphiphilic polymers comprising at least one hydrophilic unit and/or at least one fatty-chain unit;
(iv) amphoteric amphiphilic polymers comprising at least one hydrophilic unit and at least one fatty-chain unit;
   wherein the fatty chains comprise from 10 to 30 carbon atoms.

(i) The acrylic nonionic amphiphilic polymers comprising at least one fatty chain and at least one hydrophilic unit may be chosen, for example, from:
(1) copolymers of C₁-C₆ alkyl methacrylates or acrylates and of amphiphilic monomers comprising at least one fatty chain (for example oxyethylenated (C₈-C₂₂)alkyl acrylates), for instance the oxyethylenated methyl methacrylate/stearyl acrylate copolymer sold by the company Goldschmidt under the name ANTIL 208;
(2) copolymers of hydrophilic methacrylates or acrylates and of hydrophobic monomers comprising at least one fatty chain (for example (C₈-C₂₂)alkyl (meth)acrylates), for instance polyethylene glycol methacrylate/lauryl methacrylate copolymer.

(ii) The acrylic anionic amphiphilic polymers can be chosen, for example, from those comprising at least one hydrophilic unit of unsaturated olefinic carboxylic acid type, and at least one hydrophobic unit exclusively of the type such as a (C₁₀-C₃₀) alkyl ester of an unsaturated carboxylic acid. In one embodiment, the acrylic anionic amphiphilic polymers may be chosen from those in which the hydrophilic unit of unsaturated olefinic carboxylic acid type corresponds to the monomer of formula (IV) below: 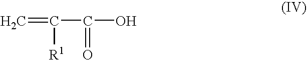
wherein R¹ is chosen from H, CH₃, and C₂H₅, i.e. acrylic acid, methacrylic acid, and ethacrylic acid units, and in which the hydrophobic unit of the type such as a (C₁₀-C₃₀) alkyl ester of an unsaturated carboxylic acid corresponds to the monomer of formula (V) below: 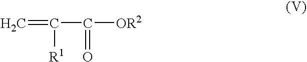
wherein R¹ is chosen from H, CH₃, and C₂H₅ (i.e. acrylate, methacrylate, and ethacrylate units) and in at least one embodiment, R¹ is chosen from H (acrylate units) and CH₃ (methacrylate units), and R² is chosen from C₁₀-C₃₀, for example, C₁₂-C₂₂ alkyl radicals.

(C₁₀-C₃₀)Alkyl esters of unsaturated carboxylic acids in accordance with the present disclosure may include, for example, lauryl acrylate, stearyl acrylate, decyl acrylate, isodecyl acrylate, and dodecyl acrylate, and the corresponding methacrylates, lauryl methacrylate, stearyl methacrylate, decyl methacrylate, isodecyl methacrylate, and dodecyl methacrylate.

Anionic amphiphilic polymers of this type are disclosed and may be prepared, for example, according to U.S. Pat. Nos. 3,915,921 and 4,509,949.

The anionic amphiphilic polymers that can be used in the context of the present disclosure may, in at least one embodiment, be chosen from polymers formed from a mixture of monomers comprising:

(i) acrylic acid and at least one ester of formula (VI) below: 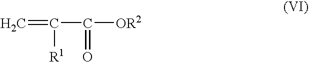
wherein R¹ is chosen from H and CH₃, and R² is chosen from alkyl radicals comprising from 12 to 22 carbon atoms, and crosslinking agents, for example, those comprising from 60% to 95% by weight of acrylic acid (hydrophilic unit), 4% to 40% by weight of C₁₀-C₃₀ alkyl acrylate (hydrophobic unit), and 0% to 6% by weight of crosslinking polymerizable monomer, or 96% to 98% by weight of acrylic acid (hydrophilic unit), 1% to 4% by weight of C₁₀-C₃₀ alkyl acrylate (hydrophobic unit), and 0.1% to 0.6% by weight of crosslinking polymerizable monomer, and

(ii) essentially acrylic acid and lauryl methacrylate, such as the product formed from 66% by weight of acrylic acid and 34% by weight of lauryl methacrylate.

The crosslinking agent may be chosen, for instance, from monomers comprising a group
CH₂=C<
with at least one other polymerizable group whose unsaturated bonds are not conjugated. Mention may be made, for instance, of polyallyl ethers such as polyallylsucrose and polyallylpentaerythritol.

According to one embodiment of the present disclosure, the polymers may be chosen from the products sold by the company Goodrich under the trade names PEMULEN TR1, PEMULEN TR2, CARBOPOL 1382, and in at least one embodiment, PEMULEN TR1, and the product sold by the company S.E.P.C. under the name COATEX SX.

Other examples of anionic amphiphilic fatty-chain polymers include, but are not limited to, ethoxylated copolymers of methacrylic acid/methyl acrylate/alkyl dimethyl-meta-isopropenylbenzylisocyanate sold under the name VISCOPHOBE DB 1000 by the company Amerchol.

Further non-limiting examples of fatty-chain anionic amphiphilic polymers include those comprising at least one acrylic monomer comprising at least one sulfonic group, in free, partially, or totally neutralized form, and comprising at least one hydrophobic portion.

The hydrophobic portion present in the polymers of the present disclosure comprise, in at least one embodiment, from 8 to 22 carbon atoms, for example, from 8 to 18 carbon atoms, or from 12 to 18 carbon atoms.

According to one embodiment of the present disclosure, the sulfonic polymers may be partially or totally neutralized with at least one base chosen from mineral bases (for example, sodium hydroxide, potassium hydroxide, and aqueous ammonia) and organic bases (such as mono-, di- or triethanolamine, aminomethylpropanediols, N-methylglucamine, and basic amino acids, for instance, arginine and lysine), and mixtures of these compounds.

The sulfonic amphiphilic polymers in accordance with the present disclosure generally have a number-average molecular weight ranging from 1000 to 20 000 000 g/mol, for instance, from 20 000 to 5 000 000, or from 100 000 to 1 500 000 g/mol.

The sulfonic amphiphilic polymers according to the present disclosure may or may not be crosslinked. In at least one embodiment, the sulfonic amphiphilic polymers are crosslinked.

When the polymers are crosslinked, the at least one crosslinking agent may be chosen from polyolefinically unsaturated compounds commonly used for the crosslinking of polymers obtained by free-radical polymerization. Such compounds include, for example, divinylbenzene, diallyl ether, dipropylene glycol diallyl ether, polyglycol diallyl ethers, triethylene glycol divinyl ether, hydroquinone diallyl ether, ethylene glycol di(meth)acrylate, tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, other allyl or vinyl ethers of polyfunctional alcohols, allylic esters of phosphoric and/or vinylphosphonic acid derivatives, and mixtures of these compounds.

In at least one embodiment, the at least one cross-linking agent may be chosen from methylenebisacrylamide, allyl methacrylate, and trimethylolpropane triacrylate (TMPTA). The degree of crosslinking may generally range from 0.01 mol % to 10 mol %, for instance, from 0.2 mol % to 2 mol % relative to the total moles of polymer.

The acrylic monomers comprising (a) at least one sulfonic group may be chosen, for example, from (meth)acrylamido(C₁-C₂₂)alkylsulfonic acids and N-(C₁-C₂₂)alkyl(meth)acrylamido(C₁-C₂₂)alkylsulfonic acids, for instance undecyl-acrylamidomethanesulfonic acid, and partially or totally neutralized forms thereof.

In one embodiment, the acrylic monomers comprising (a) at least one sulfonic group may be chosen from (meth)acrylamido(C₁-C₂₂)alkylsulfonic acids, for example, acrylamidomethanesulfonic acid, acrylamidoethanesulfonic acid, acrylamidopropanesulfonic acid, 2-acrylamido-2-methylpropanesulfonic acid, methacrylamido-2-methylpropanesulfonic acid, 2-acrylamido-n-butanesulfonic acid, 2-acrylamido-2,4,4-trimethylpentanesulfonic acid, 2-methacrylamidododecylsulfonic acid, 2-acrylamido-2,6-dimethyl-3-heptanesulfonic acid, and partially or totally neutralized forms thereof.

In another embodiment, the acrylic monomers comprising (a) at least one sulfonic group may be chosen from 2-acrylamido-2-methylpropanesulfonic acid (AMPS), and partially or totally neutralized forms thereof.

The amphiphilic polymers in accordance with the present disclosure may be chosen, for example, from random amphiphilic AMPS polymers modified by reaction with a C₆-C₂₂ n-monoalkylamine or di-n-alkylamine, such as those described, for instance, in International Patent Application Publication No. WO 00/31154, which is incorporated herein by reference in its entirety. These polymers may also comprise other ethylenically unsaturated hydrophilic monomers chosen, for example, from (meth)acrylic acids, β-substituted alkyl derivatives thereof and esters thereof obtained with monoalcohols or mono- or polyalkylene glycols, (meth)acrylamides, vinylpyrrolidone, maleic anhydride, itaconic acid, and maleic acid, and mixtures of these compounds.

According to one embodiment, the polymers of the present disclosure may be chosen, for example, from amphiphilic copolymers of AMPS and of at least one ethylenically unsaturated hydrophobic monomer comprising at least one hydrophobic portion comprising from 8 to 50 carbon atoms, for example, from 8 to 22 carbon atoms, from 8 to 18 carbon atoms, or from 12 to 18 carbon atoms.

These copolymers may also comprise at least one ethylenically unsaturated monomer not comprising a fatty chain, such as (meth)acrylic acids, β-substituted alkyl derivatives thereof or esters thereof obtained with monoalcohols, or mono- or polyalkylene glycols, (meth)acrylamides, vinylpyrrolidone, maleic anhydride, itaconic acid, or maleic acid, and mixtures thereof.

These copolymers are described, for example, in European Patent Application No. 0 750 899, U.S. Pat. No. 5,089,578, and in the following publications from Yotaro Morishima:

“Self-assembling amphiphilic polyelectrolytes and their nanostructures”—Chinese Journal of Polymer Science Vol.18, No. 40, (2000), 323-336;

“Micelle formation of random copolymers of sodium 2-(acrylamido)-2-methylpropanesulfonate and a nonionic surfactant macromonomer in water as studied by fluorescence and dynamic light scattering”—Macromolecules 2000, Vol. 33, No. 10-3694-3704;

“Solution properties of micelle networks formed by nonionic moieties covalently bound to a polyelectrolyte: salt effects on rheological behavior”—Langmuir, 2000, Vol. 16, No. 12, 5324-5332; and

“Stimuli responsive amphiphilic copolymers of sodium 2-(acrylamido)-2-methylpropanesulfonate and associative macromonomers”—Polym. Preprint, Div. Polym. Chem. 1999, 40(2), 220-221.

The ethylenically unsaturated hydrophobic monomers of these copolymers may be chosen, for example, from the acrylates and acrylamides of formula (I): 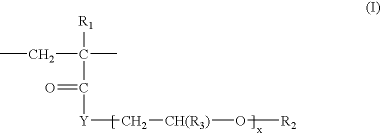
wherein:
R₁ and R₃, which may be identical or different, are chosen from hydrogen and linear or branched C₁-C₆ alkyl radicals (for example, methyl);
Y is chosen from O and NH;
R₂ is chosen from hydrophobic hydrocarbon-based radicals comprising from 8 to 50 carbon atoms, for example, from 8 to 22 carbon atoms, from 6 to 18 carbon atoms, or from 12 to 18 carbon atoms; and
x is a number ranging from 1 to 10, denoting the number of moles of alkylene oxide.

According to one embodiment, the radical R₂ may be chosen, for instance, from linear C₆-C₁₈ alkyl radicals (for example, n-hexyl, n-octyl, n-decyl, n-hexadecyl, and n-dodecyl) and branched or cyclic C6-C18 alkyl radicals (for example, cyclododecane (C₁₂) and adamantane (C₁₀)); C₆-C₁₈ alkylperfluoro radicals (for example, groups of formula —(CH₂)₂—(CF₂)₉—CF₃); cholesteryl radicals (C₂₇), cholesterol ester residues, for instance, the cholesteryl oxyhexanoate group; aromatic polycyclic groups, for instance, naphthalene and pyrene. In at least one embodiment, the radical R₂ may be chosen from linear alkyl radicals, for example, n-dodecyl radicals.

According to another embodiment of the present disclosure, the monomer of formula (I) comprises at least one alkylene oxide unit (x≧1) and optionally a polyoxyalkylenated chain. The polyoxyalkylenated chain may comprise ethylene oxide units and/or propylene oxide units, and in at least one embodiment, comprises ethylene oxide units. The number of oxyalkylene units generally ranges from 3 to 100, for example, from 3 to 50, or from 7 to 25.

Non-limiting examples of these polymers include:
crosslinked or non-crosslinked, neutralized or non-neutralized copolymers comprising from 15% to 60% by weight of AMPS units and from 40% to 85% by weight of units chosen from (C₈-C₁₆)alkyl(meth)acrylamide units and (C₈-C₁₆)alkyl (meth)acrylate units relative to the total weight of the polymer, such as those described in European Patent Application No. 0 750 899; and
terpolymers comprising from 10 mol % to 90 mol % of acrylamide units, from 0.1 mol % to 10 mol % of AMPS units, and from 5 mol % to 80 mol % of n-(C₆-C₁₈)alkylacrylamide units, such as those described in U.S. Pat. No. 5,089,578.

Further examples include, but are not limited to, copolymers of totally neutralized AMPS and of dodecyl methacrylate, and also crosslinked and non-crosslinked copolymers of AMPS and of n-dodecylmethacrylamide, such as those described in the Morishima articles mentioned above.

In at least one embodiment, the polymers may be chosen from copolymers comprising
(a) 2-acrylamido-2-methylpropanesulfonic acid (AMPS) units of formula (II): 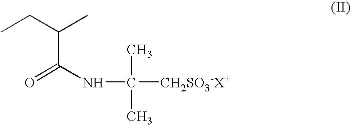
   wherein X⁺ is chosen from protons, alkali metal cations, alkaline-earth metal cations, and ammonium ions, and
(b) units of formula (III): 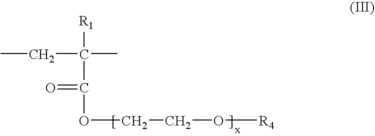
   wherein:
x is an integer ranging from 3 to 100, for example, from 5 to 80, or from 7 to 25;
R₁ has the same meaning as that given above in formula (I); and
R₄ is chosen from linear or branched C₆-C₂₂, for example, C₁₀-C₂₂ alkyl radicals.

In at least one embodiment, in formula (III), x=25, R₁ is methyl, and R₄ is n-dodecyl; these copolymers are described, for example, in the Morishima articles mentioned above.

According to another embodiment, in formula (II), X⁺ may be chosen from sodium and ammonium.

(iii) The cationic amphiphilic polymers used in the present disclosure may be chosen, for example, from polyacrylates comprising amine side groups.

The polyacrylates comprising quaternized or non-quaternized amine side groups may comprise, for example, hydrophobic groups of the type such as Steareth-20 (polyoxyethylenated (20) stearyl alcohol) and (C₁₀-C₃₀)alkyl PEG-20 itaconate.

Examples of polyacrylates comprising amino side chains include, but are not limited to, the polymers 8781-124B and 9492-103 and STRUCTURE PLUS from the company National Starch.

(iv) Suitable amphoteric amphiphilic polymers comprising at least one fatty chain, may include, for example, copolymers of methacrylamidopropyltrimethylammonium chloride/acrylic acid/C₁₀-C₃₀ alkyl methacrylate, wherein, in at least one embodiment, the alkyl radical is a stearyl radical.
(b) Examples of crosslinked acrylic acid homopolymers include, but are not limited to, those crosslinked with an allylic alcohol ether of the sugar series, for example, the products sold under the names CARBOPOL 980, 981, 954, 2984, and 5984 by the company Goodrich and the products sold under the names SYNTHALEN M and SYNTHALEN K by the company 3 VSA.
(c) Non-limiting examples of crosslinked copolymers of (meth)acrylic acid and of C₁-C₆ alkyl acrylate include the product sold under the name VISCOATEX 538C by the company Coatex, which is a crosslinked copolymer of methacrylic acid and of ethyl acrylate as an aqueous dispersion containing 38% active material, and the product sold under the name ACULYN 33 by the company Rohm & Haas, which is a crosslinked copolymer of acrylic acid and of ethyl acrylate as an aqueous dispersion containing 28% active material. For example, the crosslinked methacrylic acid/ethyl acrylate copolymer in the form of an aqueous 30% dispersion manufactured and sold under the name CARBOPOL Aqua SF-1 by the company Noveon may be used.
(d) Suitable nonionic homopolymers and copolymers containing ethylenically unsaturated monomers of ester and/or amide type may be chosen, for example, from the products sold under the names: CYANAMER P250 by the company Cytec (polyacrylamide); PMMA MBX-8C by the company US Cosmetics (methyl methacrylate/ethylene glycol dimethacrylate copolymer); ACRYLOID B66 by the company Rohm & Haas (butyl methacrylate/methyl methacrylate copolymer); and BPA 500 by the company Kobo (polymethyl methacrylate).
(e) Ammonium acrylate homopolymers may include, but are not limited to, the product sold under the name MICROSAP PAS 5193 by the company Hoechst.

Examples of copolymers of ammonium acrylate and of acrylamide that may be mentioned include, but are not limited to, the product sold under the name BOZEPOL C NOUVEAU and the product PAS 5193 sold by the company Hoechst (which are described and prepared, for example, in French Patent No. 2 416 723 and U.S. Pat. Nos. 2,798,053 and 2,923,692).
(f) Poly(meth)acrylamido(C₁ -C₄)alkylsulfonic acids

According to one embodiment, the poly(meth)acrylamido(C₁-C₄)alkylsulfonic acids may be crosslinked.

According to another embodiment, they may be partially or totally neutralized. In a further embodiment, they may be chosen from water-soluble and water-swellable polymers.

Non-limiting examples of these polymers include:
polyacrylamidomethanesulfonic acid,
polyacrylamidoethanesulfonic acid,
polyacrylamidopropanesulfonic acid,
poly-2-acrylamido-2-methylpropanesulfonic acid,
poly-2-methylacrylamido-2-methylpropanesulfonic acid, and
poly-2-acrylamido-n-butanesulfonic acid.

Polymers of this type, for instance, crosslinked and partially or totally neutralized poly-2-acrylamido-2-methylpropanesulfonic acids are described and prepared, for example, in German Patent No. 196 25 810.

These polymers generally comprise, randomly distributed:
a) from 90% to 99.9% by weight of units of formula (I): 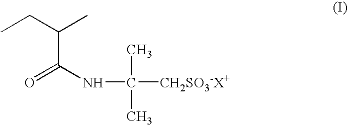
   wherein X⁺ is chosen from cations and mixtures of cations, including H⁺,
b) from 0.01% to 10% by weight of at least one crosslinking unit comprising at least two olefinic double bonds,
   wherein the weight proportions are defined relative to the total weight of the polymer; and X⁺ is chosen from cations and mixtures of cations chosen, for example, from protons, alkali metal cations, cation equivalents to that of an alkaline-earth metal, and ammonium ions.

The crosslinked and neutralized poly-2-acrylamido-2-methylpropanesulfonic acid may comprise, in at least one embodiment, from 98% to 99.5% by weight of units of formula (I) and from 0.5% to 2% by weight of crosslinking units.

The at least one crosslinking unit comprising at least two olefinic double bonds may be chosen, for example, from dipropylene glycol diallyl ether, polyglycol diallyl ethers, triethylene glycol divinyl ether, hydroquinone diallyl ether, tetraallyloxyethane or other polyfunctional alcohol allyl or vinyl ethers, tetraethylene glycol diacrylate, triallylamine, trimethylolpropane diallyl ether, methylenebisacrylamide, and divinylbenzene.

According to one embodiment, the at least one crosslinking unit comprising at least two olefinic double bonds may be chosen from those corresponding to formula (II): 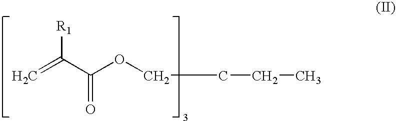
wherein R₁ is chosen from hydrogen and C₁-C₄ alkyl radicals, for instance, methyl radicals (trimethylolpropane triacrylate).

The crosslinked and partially or totally neutralized poly(2-acrylamido-2-methylpropanesulfonic acids) are generally known under the names “Ammonium polyacrylamido-2-methylpropanesulfonate” or “Ammonium polyacryidimethyltauramide” (INCI name).

A non-limiting example of a suitable commercial product is the one sold by the company Clariant under the trade name HOSTACERIN AMPS; this is a crosslinked poly(2-acrylamido-2-methylpropanesulfonic acid) partially neutralized with aqueous ammonia.
(g) The crosslinked polymers of methacryloyloxy(C₁-C₄)alkyltri(C₁-C₄)alkylammonium salts such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with an olefinically unsaturated compound, for example, methylenebisacrylamide. The cross-linked polymer may be chosen, for example, from acrylamide/methacryloyloxyethyltrimethylammonium chloride copolymers (20/80 by weight) in the form of a dispersion containing 50% by weight of the said copolymer in mineral oil. This dispersion is sold under the name “Salcare® SC 92” by the company Ciba. A crosslinked methacryloyloxyethyltrimethylammonium chloride homopolymer containing about 50% by weight of the homopolymer in mineral oil or in a liquid ester may also be used. These dispersions are sold under the names “Salcare® SC 95” and “Salcare® SC 96” by the company Ciba.

According to the present disclosure, the at least one acrylic thickening polymer may be present in the composition in an amount ranging from 0.001% to 20% by weight, for example, from 0.01 % to 10% by weight, or from 0.1% to 3% by weight relative to the total weight of the final composition.

Liquid Esters

The compositions according to the present disclosure comprise at least one liquid ester of a carboxylic acid comprising from 4 to 6 carbon atoms and of an alcohol comprising from 12 to 26 carbon atoms. These esters are insoluble in water at a concentration of greater than 0.1% at 25° C. They are liquid at 25° C. (1 atm).

The liquid esters according to the present disclosure may be chosen from those of formula (I):
R₁COOR₂ (I)
wherein:
R₁ is chosen from linear or branched, optionally mono- or polyhydroxylated hydrocarbon-based radicals comprising from 3 to 5 carbon atoms,
R₂ is chosen from linear or branched, optionally mono- or polyhydroxylated hydrocarbon-based radicals comprising from 12 to 26 carbon atoms, for example, from 16 to 22 carbon atoms.

In at least one embodiment, R₁ may be chosen from branched alkyl radicals comprising from 3 to 5 carbon atoms, for example, tert-butyl radicals.

According to another embodiment, R₂ may be chosen from saturated or unsaturated alkyl radicals comprising from 12 to 26 carbon atoms, for instance, branched radicals such as tridecyl, isocetyl, isostearyl, octyidodecyl, and isoarachidyl radicals.

In a further embodiment, the branched liquid esters may be chosen from isostearyl neopentanoate (formula (I) wherein R₁=tert-butyl and R₂=isostearyl), tridecyl neopentanoate, isocetyl neopentanoate, and isoarachidyl neopentanoate.

The at least one liquid ester may be present in the compositions in accordance with the present disclosure in an amount ranging from 0.1% to 20%, for example, from 0.2% to 10%, or from 0.5% to 5% by weight relative to the total weight of the composition.

Cyclodextrins

The compositions of the present disclosure include at least one cyclodextrin or derivative thereof. The cyclodextrins may be chosen, for example, from oligosaccharides of the following formula: 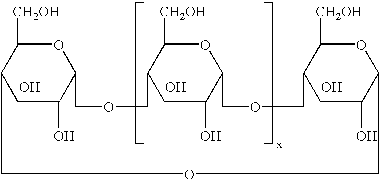
wherein x is a number ranging from 4 to 6, for example, equal to 4 (which corresponds to α-cyclodextrin), equal to 5 (β-cyclodextrin), and equal to 6 (γ-cyclodextrin).

In one embodiment, the at least one cyclodextrin may be chosen from β-cyclodextrins sold by the company Wacker under the name CAVAMAX W7 and γ-cyclodextrins sold by the company Wacker under the name CAVAMAX W8.

The cyclodextrin derivatives may include, for example, methylcyclodextrins such as the methyl-β-cyclodextrin sold by the company Wacker under the name CAVASOL W7M, and hydroxypropyl cyclodextrins, for instance, the hydroxypropyl-β-cyclodextrin sold under the name CAVASOL W7 HP by the company Wacker.

The at least one cyclodextrin may be present in the composition in an amount ranging from 1% to 15% by weight, for example, from 1% to 10% by weight, or from 1.5% to 5% by weight relative to the total weight of the final composition.

Anionic Surfactants

The compositions of the present disclosure also comprise at least one anionic surfactant, which, in at least one embodiment, is present in an amount ranging from 4% to 35%, such as from 8% to 30% relative to the total weight of the composition.

In one embodiment, the at least one cyclodextrin or derivative thereof and the at least one surfactant may be present in the composition in an amount that is effective to make the composition nacreous and/or to form an insoluble complex in the composition between the at least one cyclodextrin and the at least one surfactant.

According to another embodiment, the cyclodextrin may be introduced into the composition in non-complexed form or, in yet another embodiment, complexed with the at least one surfactant, i.e. when a complex is formed, it is not formed with a compound other than the at least one surfactant.

In at least one embodiment, the at least one cyclodextrin is complexed with the at least one surfactant.

The surfactant/cyclodextrin ratio may range from 0.01 to 300, for example, from 0.1 to 100, or from 0.3 to 25.

In the context of the present disclosure, the nature of the at least one anionic surfactant is not a critical feature.

Thus, examples of suitable anionic surfactants include, but are not limited to, salts (for example, alkaline salts, such as sodium salts, ammonium salts, amine salts, amino alcohol salts, and magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates, and N-acyltaurates, and mixtures thereof, wherein the alkyl or acyl radical of these compounds comprises, for example, from 8 to 24 carbon atoms, and the aryl radical is chosen, for instance, from phenyl and benzyl groups. Non-limiting examples of anionic surfactants include fatty acid salts such as the salts of oleic, ricinoleic, palmitic and stearic acids, coconut oil acid,.and hydrogenated coconut oil acid; and acyl lactylates wherein the acyl radical comprises from 8 to 20 carbon atoms. Weakly anionic surfactants can also be used, such as alkyl-D-galactosiduronic acids and their salts, and polyoxyalkylenated (C₈-C₂₄) alkyl ether carboxylic acids, polyoxyalkylenated (C₈-C₂₄) alkylaryl ether carboxylic acids, polyoxyalkylenated (C₈-C₂₄) alkylamido ether carboxylic acids and their salts, for example, those comprising from 2 to 50 ethylene oxide groups, and mixtures thereof.

According to one embodiment, the at least one anionic surfactant may be chosen from alkyl sulfate salts, alkyl ether sulfate salts, and mixtures thereof.

Additional Surfactants

In addition to the at least one anionic surfactant, the composition may further comprise at least one additional surfactant chosen from nonionic, amphoteric, and cationic surfactants. In at least one embodiment, the at least one additional surfactant is chosen from amphoteric and/or non-ionic surfactants.

Nonionic Surfactants

The at least one nonionic additional surfactant may be chosen from compounds that are known in the art (see, for example, “Handbook of Surfactants” by M. R. Porter, published by Blackie & Son (Glasgow and London), 1991, pp. 116-178) and, in the context of the present disclosure, their nature is not a critical feature. Thus, they can be chosen, for example, from polyethoxylated, polypropoxylated, and polyglycerolated fatty acids, alkylphenols, α-diols, and alcohols, all of these compounds having a fatty chain comprising, for example, from 8 to 18 carbon atoms, it being possible for the number of ethylene oxide or propylene oxide groups to range, for example, from 2 to 50 and for the number of glycerol groups to range, for instance, from 2 to 30. Further examples include, but are not limited to, copolymers of ethylene oxide and of propylene oxide, condensates of ethylene oxide and of propylene oxide with fatty alcohols; polyethoxylated fatty amides comprising, for instance, from 2 to 30 mol of ethylene oxide, polyglycerolated fatty amides comprising from 1 to 5, for example, from 1.5 to 4, glycerol groups; oxyethylenated fatty acid esters of sorbitan comprising from 2 to 30 mol of ethylene oxide; fatty acid esters of sucrose, fatty acid esters of polyethylene glycol, alkylpolyglycosides, N-alkylglucamine derivatives, and amine oxides such as (C₁₀-C₁₄)alkylamine oxides and N-acylaminopropylmorpholine oxides. In at least one embodiment, the non-ionic surfactants may be chosen from alkylpolyglycosides.

Amphoteric and Zwitterionic Surfactants:

The amphoteric and zwitterionic additional surfactants, whose nature is not a critical feature in the context of the present disclosure., can be chosen, for example, from aliphatic secondary or tertiary amine derivatives in which the aliphatic radical is a linear or branched chain comprising from 8 to 18 carbon atoms and comprising at least one water-soluble anionic group (for example, carboxylate, sulfonate, sulfate, phosphate or phosphonate); (C₈-C₂₀)alkylbetaines, sulfobetaines, (C₈-C₂₀)alkylamido(C₁-C₆)alkylbetaines, and/or (C₈-C₂₀)alkylamido(C₁-C₆)alkylsulfobetaines may also be used.

Examples of suitable amine derivatives include, but are not limited to, the products sold under the name MIRANOL, as described, for instance, in U.S. Pat. Nos. 2,528,378 and 2,781,354 and classified in the CTFA dictionary, 3^{rd} edition, 1982 under the names Amphocarboxy glycinates and Amphocarboxy propionates and having the respective structures:
R₂—CONHCH₂CH₂—N(R₃)(R₄)(CH₂COO—) (2)
wherein:
R₂ is chosen from alkyl radicals derived from an acid R₂—COOH present in hydrolyzed coconut oil, heptyl radicals, nonyl radicals, and undecyl radicals,
R₃ is chosen from β-hydroxyethyl groups, and
R₄ is chosen from carboxymethyl groups; and
   R_{2′}—CONHCH₂CH₂—N(B)(C) (3)
   wherein:
B is —CH₂CH₂OX′,
C is —(CH₂)_{z}—Y′, wherein z=1 or 2,
X′ is chosen from —CH₂CH₂—COOH groups and hydrogen,
Y′ is chosen from —COOH and —CH₂—CHOH—SO₃H radicals, and
R₂ is chosen from alkyl radicals of an acid R₉—COOH present in coconut oil or in hydrolyzed linseed oil, alkyl radicals, for example, C₇, C₉, C₁₁, and C₁₃ alkyl radicals, C₁₇ alkyl radicals and their iso forms, and unsaturated C₁₇ radicals.

A non-limiting example of a commercially available amine derivative is, the cocoamphodiacetate sold under the trade name MIRANOL C2M concentrated NP by the company Rhodia Chimie.

Cationic Surfactants

The cationic surfactants may be chosen, for example, from:

A) quaternary ammonium salts of formula (XII): 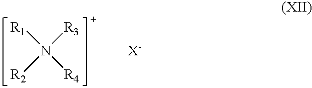
wherein
X⁻ is an anion chosen from halides (for instance, chloride, bromide, and iodide), (C₂-C₆)alkyl sulfates, such as methyl sulfate, phosphates, alkyl sulfonates, alkylaryl sulfonates, and anions derived from organic acid, such as acetate and lactate, and

i) the radicals R₁ to R₃, which may be identical or different, are chosen from linear or branched aliphatic radicals comprising from 1 to 4 carbon atoms, and aromatic radicals such as aryl and alkylaryl radicals. The aliphatic radicals may comprise at least one hetero atom such as oxygen, nitrogen, and sulphur, and/or at least one halogen. The aliphatic radicals may be chosen, for example, from alkyl, alkoxy, and alkylamide radicals, and

R₄ is chosen from linear or branched alkyl radicals comprising from 16 to 30 carbon atoms.

In at least one embodiment, the cationic surfactant is chosen from cetyl-trimethylammonium salts (for example, chloride).

ii) the radicals R₁ and R₂, which may be identical or different, are chosen from linear or branched aliphatic radicals comprising from 1 to 4 carbon atoms, and aromatic radicals such as aryl and alkylaryl radicals. The aliphatic radicals may comprise hetero atoms such as oxygen, nitrogen, and sulphur, and/or at least one halogen. The aliphatic radicals may be chosen, for example, from alkyl, alkoxy, alkylamide, and hydroxyalkyl radicals comprising from 1 to 4 carbon atoms;

R₃ and R₄, which may be identical or different, are chosen from linear or branched alkyl radicals comprising from 12 to 30 carbon atoms, wherein the radical comprises at least one function chosen from ester and amide functions;

In at least one embodiment, R₃ and R₄ may be chosen from (C₁₂-C₂₂)alkylamido(C₂-C₆)alkyl radicals and (C₁₂-C₂₂)alkylacetate radicals.

According to another embodiment, the at least one cationic surfactant may be chosen from stearamidopropyldimethyl(myristyl acetate)ammonium salts (for example, chloride).

B) quaternary ammonium salts of imidazolinium, for example, those of formula (XIII): 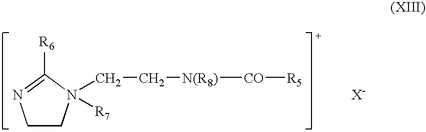
wherein:
R₅ is chosen from alkenyl and alkyl radicals comprising from 8 to 30 carbon atoms, for example, fatty acid derivatives of tallow,
R₆ is chosen from hydrogen, C₁-C₄ alkyl radicals, and alkenyl and alkyl radicals comprising from 8 to 30 carbon atoms,
R₇ is chosen from C₁-C₄ alkyl radicals,
R₈ is chosen from hydrogen, C₁-C₄ alkyl radicals, and
X is an anion chosen from halides, phosphates, acetates, lactates, alkyl sulfates, alkyl sulfonates, and alkylaryl sulfonates.

In one embodiment, R₅ and R₆ are chosen from mixtures of alkenyl and alkyl radicals comprising from 12 to 21 carbon atoms, such as, for example, fatty acid derivatives of tallow, R₇ is methyl, and R₈ is hydrogen. Examples of commercial products include, for example, Quaternium-27 (CTFA 1997) and Quaternium-83 (CTFA 1997), which are sold under the names “Rewoquat” W75, W90, W75PG, and W75HPG by the company Witco,

C) diquaternary ammonium salts of formula (XIV): 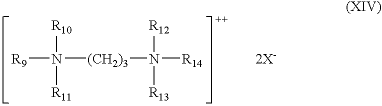
wherein:
R₉ is chosen from aliphatic radicals comprising about from 16 to 30 carbon atoms,
R₁₀, R₁₁, R₁₂, R₁₃, and R₁₄, which may be identical or different, are chosen from hydrogen and alkyl radicals comprising from 1 to 4 carbon atoms, and
X is an anion chosen from halides, acetates, phosphates, nitrates, and methyl sulfates. A non-limiting example of such a diquaternary ammonium salt is propanetallowdiammmonium dichloride.

D) quaternary ammonium salts comprising at least one ester function, of formula (XV): 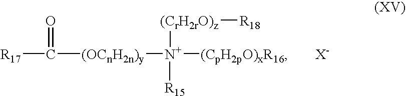
wherein:
R₁₅ is chosen from C₁-C₆ alkyl radicals, C₁-C₆ hydroxyalkyl radicals, and C₁-C₆ dihydroxyalkyl radicals;
R₁₆ is chosen from: 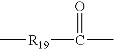
   radicals,
   linear or branched, saturated or unsaturated C₁-C₂₂ hydrocarbon-based radicals R₂₀, and
   hydrogen,
R₁₈ is chosen from: 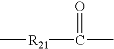
   radicals,
   linear or branched, saturated or unsaturated C₁-C₆ hydrocarbon-based radicals R₂₂, and
   hydrogen,
R₁₇, R₁₉, and R₂₁, which may be identical or different, are chosen from linear or branched, saturated or unsaturated C₇-C₂₁ hydrocarbon-based radicals;
n, p, and r, which may be identical or different, are integers ranging from 2 to 6;
y is an integer ranging from 1 to 10;
x and z, which may be identical or different, are integers ranging from 0 to 10;
X⁻ is a simple or complex, organic or inorganic anion;
   with the provisos that the sum x+y+z ranges from 1 to 15, that when x is 0, then R₁₆ is R₂₀, and that when z is 0, then R₁₈ is R₂₂.

According to one embodiment, the at least one cationic surfactant is chosen from ammonium salts of formula (XV), wherein:
R₁₅ is chosen from methyl and ethyl radicals,
x and y are equal to 1;
z is equal to 0 or 1;
n, p, and r are equal to 2;
R₁₆ is chosen from: 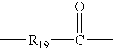
   radicals,
   methyl radicals, ethyl radicals, and C₁₄-C₂₂ hydrocarbon-based radicals, and
   hydrogen;
R₁₇, R₁₉, and R₂₁, which may be identical or different, are chosen from linear or branched, saturated or unsaturated C₇-C₂₁ hydrocarbon-based radicals;
R₁₈ is chosen from: 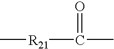
   radicals, and
   hydrogen.

Such compounds are sold, for example, under the names DEHYQUART by the company Cognis, STEPANQUAT by the company Stepan, NOXAMIUM by the company Ceca, and REWOQUAT WE 18 by the company Degussa-Witco.

According to at least one embodiment, the quaternary ammonium salts may be chosen from cetyltrimethylammonium chloride and palmitamidopropyltrimethylammonium chloride sold under the name VARISOFT PA TC by the company Degussa.

In another embodiment, the at least one anionic surfactant may be chosen from sodium, triethanolamine, and ammonium (C₁₂-C₁₄)alkyl sulfates, sodium, triethanolamine, and ammonium (C₁₂-C₁₄)alkyl ether sulfates oxyethylenated with 2.2 mol of ethylene oxide, sodium cocoyl isethionate, sodium α-(C₁₄-C₁₆)olefin sulfonate, and mixtures thereof, with an amphoteric surfactant chosen from:
amphoteric surfactants such as the amine derivatives known as disodium cocoamphodiacetate and sodium cocoamphopropionate sold, for example, by the company Rhodia Chimie under the trade name “Miranol® C2M CONCNP” as an aqueous solution containing 38% active material, or under the name Miranol® C32;
amphoteric surfactants such as alkylbetaines, for example, the cocobetaine sold under the name “Dehyton® AB 30” as an aqueous solution containing 32% AM by the company Cognis, and (C₈-C₂₀)alkylamido(C₁-C₆)alkylbetaines, for instance, Tegobetaine® F 50 sold by the company Degussa.

According to one embodiment of the present disclosure, the cosmetic compositions may further comprise agents for conditioning keratin materials.

When these compositions are applied to the hair, they may have good hair conditioning properties, i.e. the treated hair is smooth, disentangles easily, and feels soft.

The treated hair may have a natural, unladen appearance.

When the composition comprises at least one conditioning agent, the conditioning agent(s) may be chosen from synthetic oils such as poly-α-olefins, fluoro oils, fluoro waxes, fluoro gums, carboxylic acid fatty esters other than those of the present disclosure (i.e., liquid esters of a carboxylic acid comprising from 4 to 6 carbon atoms and of an alcohol comprising from 12 to 26 carbon atoms), cationic polymers, silicones, mineral, plant oils, animal oils, ceramides, pseudoceramides, and mixtures thereof.

The polyolefins may be chosen from poly-α-olefins such as
hydrogenated or non-hydrogenated polybutene polyolefins, for example, hydrogenated or non-hydrogenated polyisobutene. In at least one embodiment, the polyolefins may be chosen from isobutylene oligomers with a molecular weight of less than 1000 and mixtures thereof with polyisobutylenes with a molecular weight of greater than 1000, for example, ranging from 1000 to 15 000.

Non-limiting examples of poly-α-olefins include polyisobutenes sold under the name PERMETHYL 99 A, 101 A, 102 A, 104 A (n=16), and 106 A (n=38) by the company Presperse Inc., and the products sold under the name ARLAMOL HD (n=3) by the company ICI (n denoting the degree of polymerization), and
hydrogenated or non-hydrogenated polydecene polyolefins.

Such products are sold, for example, under the names ETHYLFLO by the company Ethyl Corp. and ARLAMOL PAO by the company ICI. The mineral oils that may be used in the compositions of the present disclosure may be chosen, for example, from hydrocarbons, such as hexadecane, and liquid paraffin.

The cationic polymers that may be used in accordance with the present disclosure may be chosen from those known in the art as improving the cosmetic properties of hair treated with detergent compositions, for instance, those described in European Patent Application No. 0 337 354 and French Patent Application Nos. 2 270 846, 2 383 660, 2 598 611, 2 470 596, and 2 519 863.

As used herein, the term “cationic polymer” denotes any polymer containing at least one cationic group and/or at least one group that may be ionized into cationic groups.

Non-limiting examples of cationic polymers that may be used in the compositions of the present disclosure include quaternary cellulose ether derivatives such as the products sold under the name “JR 400” by the company Amerchol, cyclopolymers, such as diallyldimethylammonium salt homopolymers and the copolymers of a diallyldimethylammonium salt and of acrylamide, for instance, the chlorides, sold under the names “Merquat 100”, “Merquat 550”, and “Merquat S” by the company Nalco, cationic polysaccharides such as guar gums modified with 2,3-epoxypropyltrimethylammonium chloride, sold, for example, under the name “Jaguar C13S” by the company Meyhall, and optionally crosslinked homopolymers and copolymers of a (meth)acryloyloxyethyltrimethylammonium salt, sold by the company Ciba as a 50% solution in mineral oil, under the trade names Salcare® SC92 (crosslinked copolymer of methacryloyloxyethyltrimethylammonium chloride and of acrylamide) and Salcare® SC95 (crosslinked homopolymer of methacryloyloxyethyltrimethylammonium chloride).

The cationic polymers may also be chosen from polymers comprising repeating units of formula (a): 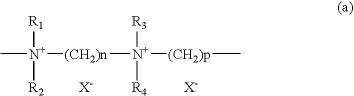
wherein:
R₁, R₂, R₃, and R4, which may be identical or different, are chosen from alkyl and hydroxyalkyl radicals comprising from 1 to 4 carbon atoms,
n and p, which may be identical or different, are integers ranging from 2 to 20, and
X⁻ is an anion derived from a mineral or organic acid.

The silicones that may be used in accordance with the present disclosure may include, for example, polyorganosiloxanes that are insoluble in the composition and that may be in a form chosen from oils, waxes, resins, and gums.

As used herein, water-insoluble silicones are silicones that are insoluble in water at a concentration of greater than or equal to 0.1% by weight in water at 25° C., i.e., they do not form a transparent isotropic solution.

The viscosity of the silicones may be measured, for example, at 25° C. according to ASTM standard 445 Appendix C. Organopolysiloxanes suitable for use in the compositions of the present disclosure are defined, for example, in Walter Noll's “Chemistry and Technology of Silicones” (1968) Academic Press. The organopolysiloxanes can be volatile or non-volatile.

When they are volatile, the silicones may be chosen from those having a boiling point ranging from 60° C. to 260° C., for example:
(i) cyclic silicones comprising from 3 to 7, for instance, from 4 to 5 silicon atoms. These silicones include, for example, octamethylcyclotetrasiloxane, such as the product sold under the name “Volatile Silicone 7207” by Union Carbide and the product “Silbione 70045 V 2” sold by Rhodia Chimie, decamethylcyclopentasiloxane, such as the product sold under the name “Volatile Silicone 7158” by Union Carbide, and the product “Silbione 70045 V 5” sold by Rhodia Chimie, and mixtures thereof.

The volatile silicones may also include cyclocopolymers of the dimethylsiloxane/methylalkylsiloxane type, such as “Silicone Volatile FZ 3109” sold by the company Union Carbide, having the chemical structure:

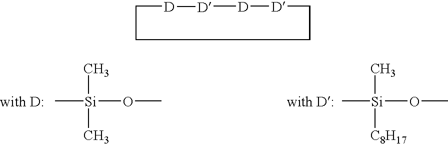

Mixtures of cyclic silicones with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane may also be used;
(ii) linear volatile silicones comprising from 2 to 9 silicon atoms and having a viscosity of less than or equal to 5×10⁻⁶ m²/s at 25° C., for example, decamethyltetrasiloxane sold, for instance, under the name “SH 200” by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Non-volatile silicones may also be used in the compositions of the present disclosure. Such silicones may be chosen from, but not limited to, polyalkylsiloxanes, polyarylsiloxanes, polyalkylarylsiloxanes, silicone gums and resins, polyorganosiloxanes modified with organofunctional groups, and mixtures thereof.

These silicones may be chosen, for example, from polyalkylsiloxanes, such as polydimethylsiloxanes comprising trimethylsilyl end groups having a viscosity ranging from 5×10⁻⁶ to 2.5 m²/s at 25° C., for instance, from 1×10⁻⁵ to 1 m²/s.

Non-limiting examples of commercially available polyalkylsiloxanes include the following commercial products:
the oils of the Mirasil series sold by the company Rhodia Chimie, such as, for example, the oil MIRASIL DM 500 000;
the oils of the 200 series from the company Dow Corning, such as, for example, DC200 having a viscosity of 60 000 cSt;
the Viscasil oils from General Electric and certain oils of the SF series (for example, SF 96 and SF 18) from General Electric.

Other examples include, but are not limited to, polydimethylsiloxanes comprising dimethylsilanol end groups (Dimethiconol according to the CTFA name) such as the oils of the 48 series from the company Rhodia Chimie. Further non-limiting examples of polyalkylsiloxanes in this category include the products sold under the names “Abil Wax 9800 and 9801” by the company Degussa, which are poly(C₁-C₂₀)alkylsiloxanes.

The polyalkylarylsiloxanes may be chosen, for instance, from linear and/or branched polydimethylmethylphenylsiloxanes and polydimethyldiphenylsiloxanes, with a viscosity ranging from 1×10⁻⁵ to 5×10⁻² m²/s at 25° C.

Non-limiting examples of commercially available polyalkylarylsiloxanes include the products sold under the following names:
the Mirasil DPDM oils from Rhodia Chimie;
the oils of the Rhodorsil 70 633 and 763 series from Rhodia Chimie;
the oil Dow Corning 556 Cosmetic Grade Fluid from Dow Coming;
the silicones of the PK series from Bayer, such as the product PK20;
the silicones of the PN and PH series from Bayer, such as the products PN1000 and PH1000; and
certain oils of the SF series from General Electric, such as SF 1023, SF 1154, SF 1250, and SF 1265.

The silicone gums that can be used in accordance with the present disclosure may include, for example, polydiorganosiloxanes having high number-average molecular masses ranging from 200 000 to 1 000 000, used alone or as a mixture in a solvent. This solvent can be chosen from volatile silicones, polydimethylsiloxane (PDMS) oils, polyphenylmethylsiloxane (PPMS) oils, isoparaffins, polyisobutylenes, methylene chloride, pentane, dodecane, tridecane, and mixtures thereof.

Further examples of silicone gums include, but are not limited to:
polydimethylsiloxane,
polydimethylsiloxane/methylvinylsiloxane gums,
polydimethylsiloxane/diphenylsiloxane,
polydimethylsiloxane/phenylmethylsiloxane, and
polydimethylsiloxane/diphenylsiloxane/methylvinylsiloxane.

Non-limiting examples of suitable mixtures of silicone gums include:
mixtures formed from a polydimethylsiloxane hydroxylated at the chain end (referred to as dimethiconol according to the nomenclature in the CTFA dictionary) and from a cyclic polydimethylsiloxane (referred to as cyclomethicone according to the nomenclature in the CTFA dictionary), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane; and
mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s, and an SF 96 oil, with a viscosity of 5×10⁻⁶ m²/s. In at least one embodiment, this product contains 15% SE 30 gum and 85% SF 96 oil.

The organopolysiloxane resins that can be used in accordance with the present disclosure may include crosslinked siloxane systems comprising at least one of the following units:
R₂SiO_{2/2}, R₃SiO_{1/2}, RSiO_{3/2}, and SiO_{4/2}, wherein R is chosen from hydrocarbon-based groups comprising from 1 to 16 carbon atoms and phenyl groups. In at least one embodiment, R may be chosen from C₁-C₄ lower alkyl radicals, such as methyl and phenyl radicals.

Examples of these resins include, but are not limited to, the product sold under the name “Dow Coming 593” and those sold under the names “Silicone Fluid SS 4230 and SS 4267” by the company General Electric, which are silicones of dimethyl/trimethyl siloxane structure.

Further non-limiting examples include the trimethyl siloxysilicate type resins sold under the names X22-4914, X21-5034, and X21-5037 by the company Shin-Etsu.

The organomodified silicones that can be used in accordance with the present disclosure include silicones, for example, those defined above, which comprise in their structure at least one organofunctional group attached via a hydrocarbon-based radical.

Non-limiting examples of such organomodified silicones may include polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils SILWET L 722, L 7500, L 77, and L 711 sold by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups may be, in at least one embodiment, chosen from C₁-C₄ aminoalkyl groups;
thiol groups such as the products sold under the names “GP 72 A” and “GP 71” from Genesee;
alkoxylated groups such as the product sold under the name “Silicone Copolymer F-755” by SWS Silicones and ABIL WAX 2428, 2434 and 2440 by the company Degussa;
hydroxylated groups such as the polyorganosiloxanes comprising a hydroxyalkyl function, described, for example, in French Patent Application No. 85 16334;
acyloxyalkyl groups such as the polyorganosiloxanes described in U.S. Pat. No. 4,957,732;
anionic groups of carboxylic type, such as those in the products described in European Patent No 0 186 507 from the company Chisso Corporation, or of alkylcarboxylic type, such as those present in the product X-22-3701 E from the company Shin-Etsu; 2-hydroxyalkyl sulfonate; 2-hydroxyalkyl thiosulfate such as the products sold by the company Degussa under the names “Abil S201” and “Abil S255”; and
hydroxyacylamino groups, such as the polyorganosiloxanes described in European Patent Application No. 0 342 834, for example, the product Q2-8413 from the company Dow Corning.

According to the present disclosure, it is also possible to use silicones comprising a polysiloxane portion and a portion comprising a non-silicone organic chain, one of the two portions constituting the main chain of the polymer, the other being grafted onto the main chain. These polymers are described, for example, in European Patent Application Nos. 0 412 704, 0 412 707, 0 640 105, and 0 582 152, International Patent Application Publication Nos. WO 95/00578 and WO 93/23009, and U.S. Pat. Nos. 4,693,935, 4,728,571, and 4,972,037. These polymers may be chosen from anionic and non-ionic polymers.

Such polymers include, for example, copolymers that can be obtained by free-radical polymerization starting with a monomer mixture comprising:
a) 50 to 90% by weight of tert-butyl acrylate;
b) 0 to 40% by weight of acrylic acid;
c) 5 to 40% by weight of silicone macromer of formula:

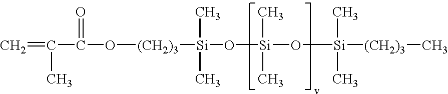

wherein v is a number ranging from 5 to 700; the weight percentages being calculated relative to the total weight of the monomers.

Other examples of grafted silicone polymers include, but are not limited to, polydimethylsiloxanes (PDMS) onto which are grafted, via a connecting chain unit of thiopropylene type, mixed polymer units of poly(meth)acrylic acid type and of polyalkyl (meth)acrylate type and polydimethylsiloxanes (PDMS) onto which are grafted, via a connecting chain unit of thiopropylene type, polymer units of polyisobutyl (meth)acrylate type.

According to the present disclosure, all of the silicones described herein may also be used in the form of emulsions.

In another embodiment, the polyorganosiloxanes may be chosen, for example, from:
non-volatile silicones chosen from the family of polyalkylsiloxanes comprising trimethylsilyl end groups, such as oils having a viscosity ranging from 0.2 to 2.5 m²/s at 25° C., such as the oils of the DC200 series from Dow Corning, for instance, that with a viscosity of 60 000 cSt, of the Mirasil DM series, the oil MIRASIL DM 500 000 sold by the company Rhodia Chimie, and the silicone oil AK 300 000 from the company Wacker, polyalkylsiloxanes comprising dimethylsilanol end groups such as dimethiconols and polyalkylarylsiloxanes, such as the oil MIRASIL DPDM sold by the company Rhodia Chimie; and
polysiloxanes comprising amine groups, such as amodimethicones and trimethylsilyl amodimethicones.

According to a further embodiment of the present disclosure, the compounds of ceramide type may be chosen from natural ceramides, synthetic ceramides. glycoceramides, pseudoceramides, and/or neoceramides.

Compounds of ceramide type are described, for example, in German Patent Application Nos. 4 424 530, 4 424 533, 4 402 929, and 4 420 736, International Patent Application Publication Nos. WO 95/23807, WO 94/07844, WO 95/16665, WO 94/07844, WO 94/24097 and WO 94/10131, French Patent Application No. 2 673 179, and European Patent Application Nos. 0 227 994 and 0 646 572, the teachings of which are incorporated herein by reference in their entireties.

Non-limiting examples of compounds of ceramide type include:
2-N-linoleoylaminooctadecane-1,3-diol,
2-N-oleoylaminooctadecane-1,3-diol,
2-N-palmitoylaminooctadecane-1,3-diol,
2-N-stearoylaminooctadecane-1,3-diol,
2-N-behenoylaminooctadecane-1,3-diol,
2-N-[2-hydroxypalmitoyl]aminooctadecane-1,3-diol,
2-N-stearoylaminooctadecane-1,3,4-triol, for example N-stearoylphytosphingosine,
2-N-palmitoylaminohexadecane-1,3-diol
bis(N-hydroxyethyl-N-cetyl)malonamide,
N-(2-hydroxyethyl)-N-(3-cetyloxy-2-hydroxypropyl)cetylamide,
N-docosanoyl-N-methyl-D-glucamine, and
mixtures thereof.

The at least one conditioning agent other than the esters of the present disclosure may be present in the composition in an amount ranging from 0.001% to 10% by weight, for example, from 0.005% to 5% by weight, or from 0.01% to 3% by weight relative to the total weight of the composition.

The physiologically acceptable medium may be chosen from water and mixtures of water and at least one cosmetically and/or dermatologically acceptable solvent such as monoalcohols, polyalcohols, glycol ethers, and mixtures thereof. The water may be present in the composition in an amount ranging from 30% to 98% by weight, for example, from 50% to 98% by weight relative to the total weight of the composition.

Examples of suitable solvents include, but are not limited to, monoalcohols such as ethanol and isopropanol, polyalcohols such as diethylene glycol and glycerol, and polyol ethers such as propylene glycol alkyl ethers.

The composition of the present disclosure may further comprise at least one optional additive chosen from sequestering agents, softeners, foam modifiers, dyes, other nacreous agents, hydrating agents, antidandruff or antiseborrheic agents, other suspension agents, hydroxy acids, thickeners, fatty acid esters, fragrances, preserving agents, sunscreens, proteins, vitamins, provitamins, polymers, and any other additive conventionally used in cosmetics.

These additives may bee present in the composition according to the present disclosure in an amount ranging from 0 to 40% by weight relative to the total weight of the composition. The amount of each additive may depend on its nature and may be readily determined by a person skilled in the art.

It is to be understood that a person skilled in the art will take care to select the at least one optional compound(s) to be added to the composition according to the disclosure such that the advantageous properties intrinsically associated with the composition in accordance with the present disclosure are not, or are not substantially, adversely affected by the envisaged addition.

The compositions according to the present disclosure may be in a form chosen from gels, milks, creams, more or less thickened lotions, and mousses.

The compositions in accordance with the present disclosure may be used for treating keratin materials such as the hair, the skin, the eyelashes, the eyebrows, the nails, the lips, and/or the scalp; in at least one embodiment, the compositions are used to treat the hair.

The compositions may also be used for washing and cleansing keratin materials such as the hair and the skin.

The compositions according to the present disclosure may generally be used as products for washing, caring for, conditioning, maintaining the hairstyle, and/or for shaping keratin materials such as the hair.

The compositions of the present disclosure may be in a form chosen from shampoos, rinse-out or leave-in conditioners, permanent-waving compositions, relaxing compositions, dyeing compositions, bleaching compositions, compositions to be applied before or after dyeing, bleaching, permanent-waving, and/or relaxing the hair, and compositions to be applied between the two steps of a permanent-waving and/or hair-relaxing operation. In at least one embodiment, the compositions are washing and foaming compositions for the hair and/or the skin.

In another embodiment, the compositions according to the present disclosure may be foaming detergent compositions such as shampoos, shower gels, and bubble baths. In this embodiment, the compositions comprise at least one detergent surfactant.

The at least one detergent surfactant may be chosen, for example, from the anionic, amphoteric, nonionic, zwitterionic, and cationic surfactants described above, and mixtures thereof, and in at least one embodiment, from anionic surfactants and mixtures of anionic surfactants and amphoteric and/or nonionic surfactants.

The amount of surfactant may vary depending on the type of surfactant and desired application; however, generally speaking, the minimum amount of surfactant is that which is just sufficient to give the final composition a satisfactory foaming power and/or detergent power.

Thus, according to the present disclosure, the at least one detergent surfactant may be present in the composition in an amount ranging from 3% to 30% by weight, for example, from 6% to 25% by weight, or from 8% to 20% by weight, relative to the total weight of the final composition.

The foaming power of the compositions according to the present disclosure, characterized by a foam height, may be greater than 75 mm, for example, greater than 100 mm, as measured according to the modified Ross-Miles method (NF T 73-404/ISO 696). The modifications to the method are as follows:

The measurement is performed at a temperature of 22° C. with osmosed water. The concentration of the solution is 2 g/l. The drop height is 1 m. The amount of composition that is dropped is 200 ml. These 200 ml of composition fall into a measuring cylinder 50 mm in diameter and containing 50 ml of the test composition. The measurement is carried out 5 minutes after stopping the flow of the composition.

The compositions of the present disclosure may also be in a form chosen from rinse-out or leave-in conditioners, permanent-waving compositions, relaxing compositions, dyeing compositions, bleaching compositions, rinse-out compositions, to be applied before or after dyeing, bleaching, permanent-waving, and/or relaxing the hair, and compositions to be applied between the two steps of a permanent-waving and/or hair-relaxing operation.

When the composition is in the form of a conditioner optionally to be rinsed out, it may comprise at least one cationic surfactant, present in an amount ranging from 0.1% to 10% by weight, for example, from 0.5% to 5% by weight relative to the total weight of the composition.

The compositions of the present disclosure may also be in the form of washing compositions for the skin, for example, bath and/or shower solutions and/or gels and makeup-removing products.

The compositions according to the present disclosure may also be in the form of aqueous or aqueous-alcoholic lotions for skincare and/or haircare.

The cosmetic compositions according to the present disclosure may be in a form chosen from gels, milks, creams, emulsions, thickened lotions, and mousses and may be used for the skin, the nails, the eyelashes, the lips, and/or the hair. In at least one embodiment, these compositions may be liquid.

The compositions may be packaged in various forms, for example, in vaporizers, pump-dispenser bottles, and aerosol containers in order to be able to apply the composition in a vaporized form or in the form of a mousse. Such packaging forms are indicated, for example, when it is desired to obtain a form chosen from sprays, lacquers, and mousses for treating the hair.

Also disclosed herein is also a cosmetic process for treating keratin materials such as the hair, which comprises applying to the keratin materials a composition as defined above, optionally followed by rinsing with water after an optional leave-in time.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contain certain errors necessarily resulting from the standard deviation found in their respective testing measurements.

By way of non-limiting illustration, concrete examples of certain embodiments of the present disclosure are given below. In the text hereinbelow, “A.M.” means Active Material.

### EXAMPLES

### Example 1

Two shampoos B and C in accordance with the present disclosure and a shampoo A (comparative composition not in accordance with the present disclosure) were prepared:

| | % AM | | |
|---|---|---|---|
| | A | | |
| | (comparative) | B | C |
| Water qs | 100 | 100 | 100 |
| Polyquaternium-10 (UCARE | 0.4 | 0.4 | 0.4 |
| POLYMER JR 400 from | | | |
| Amerchol) | | | |
| Carbomer (CARBOPOL 980 | 0.2 | 0.2 | 0.2 |
| from Noveon) | | | |
| Isostearyl neopentanoate | | 0.5 | 0.25 |
| (CERAPHYL 375 from ISP) | | | |
| β-Cyclodextrin | 2 | 2 | 2 |
| (CAVAMAX W7 from Wacker) | | | |
| Sodium lauryl ether | 14 | 14 | 14 |
| sulfate containing 2.2 | | | |
| mol of ethylene oxide | | | |
| Cocoamidopropylbetaine | 2.4 | 2.4 | 2.4 |
| Dimethicone (MIRASIL | 1.5 | 1.5 | 1.5 |
| DM 500 000 from Rhodia) | | | |
| Vitamins: (nicotinamide | 0.3 | 0.3 | 0.3 |
| from DSM, d-panthenol | | | |
| from BASF, dl-α- | | | |
| tocopheryl acetate | | | |
| from DSM) | | | |
| Preserving agent | 0.45 | 0.45 | 0.45 |
| Fragrance | qs | Qs | qs |
| Sodium hydroxide/citric | pH | pH | pH |
| acid qs | 6.8-7 | 6.8-7 | 6.8-7 |
| Viscosity (flow time in | 55 | 69 | 58 |
| seconds, C Ford 10) | | | |

Compositions B and C were stable for more than 2 months at room temperature and at 45° C. They had a beautiful nacreous effect and good cosmetic properties. Hair treated with these compositions disentangled easily and was smooth.

Composition A was unstable (presence of two phases) after 51 days at 45° C.

### Example 2

A shampoo in accordance with the present disclosure was prepared:

| | % AM |
|---|---|
| Polyquaternium-10 (UCARE POLYMER JR 400 from | 1.2 |
| Amerchol) | |
| Carbomer (CARBOPOL 980 from Noveon) | 0.2 |
| Tridecyl neopentanoate (CERAPHYL 55 from ISP) | 0.5 |
| β-Cyclodextrin (CAVAMAX W7 from Wacker) | 2 |
| Sodium lauryl ether sulfate containing 2.2 mol of | 11.2 |
| ethylene oxide | |
| Cocamide MIPA (EMPILAN CIS from Huntsmann) | 1 |
| Disodium cocoamphodiacetate (MIRANOL C2M conc. | 3 |
| from Rhodia) | |
| Quaternized wheat protein hydrolysate (Hydrotriticum | 0.3 |
| WQ1) | |
| Glucose ester of vitamin F | 0.01 |
| Glycerol ester of vitamin F | 0.1 |
| Glycerol | 1 |
| Preserving agent | 1.2 |
| Fragrance | qs |
| Sodium hydroxide/citric acid qs | pH 5-5.5 |
| Water qs | 100 |

What is claimed is:

1. A cosmetic composition comprising, in a cosmetically acceptable aqueous medium:
at least one anionic surfactant,
at least one liquid ester of a carboxylic acid comprising from 4 to 6 carbon atoms and of an alcohol comprising from 12 to 26 carbon atoms,
at least one compound chosen from cyclodextrins and derivatives thereof, and
at least one acrylic thickening polymer.

2. The composition according to claim 1, wherein the at least one compound chosen from cyclodextrins and derivatives thereof is chosen from α-cyclodextrins, β-cyclodextrins, γ-cyclodextrins, and derivatives thereof.

3. The composition according to claim 2, wherein the cyclodextrins are chosen from β-cyclodextrins and γ-cyclodextrins.

4. The composition according to claim 2, wherein the cyclodextrins are chosen from β-cyclodextrins.

5. The composition according to claim 1, wherein the at least one compound chosen from cyclodextrins and derivatives thereof is present in the composition in an amount ranging from 1% to 10% by weight relative to the total weight of the composition.

6. The composition according to claim 5, wherein the at least one compound chosen from cyclodextrins and derivatives thereof is present in the composition in an amount ranging from 1.5% to 5% by weight relative to the total weight of the composition.

7. The composition according to claim 1, wherein the at least one surfactant and the at least one compound chosen from cyclodextrins and derivatives thereof are present in the composition in concentrations that are effective to form an insoluble complex in the composition, and/or to make the composition nacreous.

8. The composition according to claim 1, wherein the at least one liquid ester is chosen from compounds of formula (I):
R₁COOR₂ (I)
wherein:
R₁ is chosen from linear or branched, optionally mono- or polyhydroxylated hydrocarbon-based radicals comprising from 3 to 5 carbon atoms, and
R₂ is chosen from linear or branched, optionally mono- or polyhydroxylated hydrocarbon-based radicals comprising from 12 to 26 carbon atoms.

9. The composition according to claim 8, wherein:
R₁ is chosen from branched alkyl radicals comprising from 3 to 5 carbon atoms, and
R₂ is chosen from optionally branched alkyl radicals comprising from 12 to 26 carbon atoms.

10. The composition according to claim 8, wherein R₁ is a tert-butyl radical.

11. The composition according to claim 8, wherein R₂ is chosen from tridecyl, isocetyl, isostearyl, octyidodecyl, and isoarachidyl radicals.

12. The composition according to claim 1, wherein the at least one liquid ester is present in the composition in an amount ranging from 0.1% to 20% relative to the total weight of the composition.

13. The composition according to claim 12, wherein the at least one liquid ester is present in the composition in an amount ranging from 0.2% to 10% by weight relative to the total weight of the composition.

14. The composition according to claim 1, wherein the at least one acrylic thickening polymer is chosen from:
(a) acrylic associative thickening polymers;
(b) crosslinked acrylic acid homopolymers;
(c) crosslinked copolymers of (meth)acrylic acid and of a (C₁-C₆)alkyl acrylate;
(d) nonionic homopolymers and copolymers comprising ethylenically unsaturated ester and/or amide monomers;
(e) ammonium acrylate homopolymers and copolymers of ammonium acrylate and of acrylamide;
(f) (meth)acrylamido(C₁-C₄)alkylsulfonic acid homopolymers and copolymers; and
(g) crosslinked methacryloyl(C₁-C₄)alkyltri(C₁-C₄)alkylammonium homopolymers and copolymers.

15. The composition according to claim 1, wherein the at least one acrylic thickening polymer is chosen from:
(a) acrylic associative thickening polymers;
(b) crosslinked acrylic acid homopolymers; and
(c) crosslinked copolymers of (meth)acrylic acid and of a (C₁-C₆)alkyl acrylate.

16. The composition according to claim 15, wherein the at least one acrylic associative thickening polymer is chosen from acrylic anionic amphiphilic polymers comprising at least one hydrophilic unit of formula (IV) and at least one hydrophobic unit chosen from (C₁₀-C₃₀)alkyl esters of an unsaturated carboxylic acid of formula (IV): 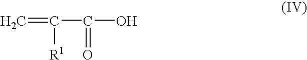
wherein R¹ is chosen from H, CH₃, and C₂H₅, and
wherein the hydrophobic unit chosen from (C₁₀-C₃₀) alkyl esters of an unsaturated carboxylic acid corresponds to the monomer of formula (V): 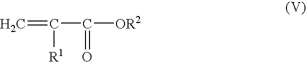
wherein R¹ is chosen from H, CH₃, and C₂H₅, and R² is chosen from C₁₀-C₃₀ alkyl radicals.

17. The composition according to claim 16, wherein in formula (V) R¹ is chosen from H and CH₃.

18. The composition according to claim 16, wherein in formula (V) R² is chosen from C₁₂-C₂₂ alkyl radicals.

19. The composition according to claim 1, wherein the at least one acrylic thickening polymer is present in the composition in an amount ranging from 0.001% to 20% by weight relative to the total weight of the composition.

20. The composition according to claim 19, wherein the at least one acrylic thickening polymer is present in the composition in an amount ranging from 0.01% to 10% by weight relative to the total weight of the composition.

21. The composition according to claim 20, wherein the at least one acrylic thickening polymer is present in the composition in an amount ranging from 0.1% to 3% by weight relative to the total weight of the composition.

22. The composition according to claim 1, wherein the at least one anionic surfactant is chosen from:
(a) salts of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates, and N-acyltaurates,
wherein the alkyl or acyl radical of these compounds comprises from 8 to 24 carbon atoms, and the aryl radical is chosen from phenyl and benzyl groups;
(b) fatty acid salts chosen from the salts of oleic, ricinoleic, palmitic, and stearic acids, coconut oil acid, and hydrogenated coconut oil acid; acyl lactylates wherein the acyl radical comprises from 8 to 20 carbon atoms; alkyl-D-galactosiduronic acids and their salts, and polyoxyalkylenated (C₈-C₂₄) alkyl ether carboxylic acids, polyoxyalkylenated (C₈-C₂₄) alkylaryl ether carboxylic acids, polyoxyalkylenated (C₈-C₂₄) alkylamido ether carboxylic acids and their salts, and
mixtures thereof.

23. The composition according to claim 22, wherein the salts (a) are chosen from alkaline salts, ammonium salts, amine salts, amino alcohol salts, and magnesium salts.

24. The composition according to claim 23, wherein the alkaline salts are sodium salts.

25. The composition according to claim 1, wherein the at least one anionic surfactant is present in the composition in an amount ranging from 4% to 35% relative to the total weight of the composition.

26. The composition according to claim 25, wherein the at least one anionic surfactant is present in the composition in an amount ranging from 8% to 30% relative to the total weight of the composition.

27. The composition according to claim 1, further comprising at least one conditioning agent.

28. The composition according to claim 27, wherein the at least one conditioning agent is chosen from poly-α-olefins, fluoro oils, fluoro waxes, fluoro gums, carboxylic acid esters other than liquid esters of a carboxylic acid comprising from 4 to 6 carbon atoms and of an alcohol comprising from 12 to 26 carbon atoms, silicones, cationic polymers, mineral oils, plant oils, animal oils, ceramides, pseudoceramides, and mixtures thereof.

29. The composition according to claim 27, wherein the at least one conditioning agent is present in the composition in an amount ranging from 0.001% to 10% by weight relative to the total weight of the composition.

30. The composition according to claim 29, wherein the at least one conditioning agent is present in the composition in an amount ranging from 0.005% to 5% by weight relative to the total weight of the composition.

31. The composition according to claim 30, wherein the at least one conditioning agent is present in the composition in an amount ranging from 0.01% to 3% by weight relative to the total weight of the composition.

32. The composition according to claim 1, wherein the composition is in a form chosen from gels, milks, creams, lotions, and mousses.

33. The composition according to claim 1, wherein the composition is a foaming detergent composition.

34. The composition according to claim 33, wherein the foaming detergent composition is chosen from shampoos, shower gels, and bubble baths.

35. The composition according to claim 1, wherein the-composition is chosen from rinse-out or leave-in hair-conditioning compositions, permanent-waving compositions, relaxing compositions, dyeing compositions, bleaching compositions, compositions to be applied before or after dyeing, bleaching, permanent-waving, and/or relaxing the hair, and compositions to be applied between the two steps of a permanent-waving and/or hair-relaxing operation.

36. A cosmetic process for treating keratin materials comprising applying a composition to the keratin materials and optionally rinsing the keratin materials with water, wherein the composition comprises, in a cosmetically acceptable aqueous medium:
at least one anionic surfactant,
at least one liquid ester of a carboxylic acid comprising from 4 to 6 carbon atoms and of an alcohol comprising from 12 to 26 carbon atoms,
at least one compound chosen from cyclodextrins and derivatives thereof, and
at least one acrylic thickening polymer.

37. A process for improving the stability on storage of a cosmetic composition comprising at least one cyclodextrin and at least one anionic surfactant, comprising combining the composition with at least one thickening acrylic polymer and at least one liquid ester of a carboxylic acid comprising from 4 to 6 carbon atoms and of an alcohol comprising from 12 to 26 carbon atoms.
